(19) United States
(12) Patent Application Publication
(10) Pub. No.: US 2018/0228728 A1
(43) Pub. Date: Aug. 16, 2018

(54) DOSAGE FORM FOR ADMINISTRATION OF OPIOID ANTAGONISTS

(71) Applicant: Kirti H. Valia, Plainsboro (PA)

(72) Inventor: Kirti H. Valia, Plainsboro (PA)

(21) Appl. No.: 15/894,377

(22) Filed: Feb. 12, 2018

Related U.S. Application Data
(60) Provisional application No. 62/504,347, filed on May 10, 2017.
(60) Provisional application No. 62/458,252, filed on Feb. 13, 2017.

(51) Int. Cl. A61K 9/00; A61K 31/485; A61K 47/10; A61K 47/34; A61K 47/46; A61K 47/44; A61K 47/38; A61K 47/18; A61K 47/12; A61P 25/36

(52) U.S. Cl. CPC A61K 9/0056; A61K 31/485; A61K 47/10; A61K 47/34; A61P 25/36; A61K 47/44; A61K 47/38; A61K 47/18; A61K 47/12; A61K 47/46

(57) ABSTRACT

A novel dosage form for delivering an opioid antagonist such as naloxone is disclosed. The dosage form comprises an oral dissolvable film containing an amount of the opioid antagonist effective to reduce or counteract the effect of an opioid overdose in an individual. Methods of administering the dosage form to an individual experiencing an opioid overdose are also disclosed.

### CROSS-REFERENCE TO RELATED APPLICATIONS

This claims benefit of U.S. Provisional Application Nos. 62/504,347, filed May 10, 2017, and 62/458,252, filed Feb. 13, 2017, the entire contents of each of which are incorporated by reference herein.

### FIELD OF THE INVENTION

Aspects of the present invention relate generally to pharmaceutical compositions, and more particularly to a dosage form for administration of opioid antagonists, in particular naloxone.

### BACKGROUND OF THE INVENTION

Various publications, including patents, published applications and scholarly articles, are cited throughout the specification. Each of these publications is incorporated by reference herein, in its entirety.

Opioid overdoses have become an international health crisis. It has been estimated that, in 2014, there were 113,700-250,100 drug-related deaths worldwide, with overdose accounting for up to half of all deaths and with opioids involved in most cases (see The United Nations Office on Drugs and Crime (UNODC) ‘2016 World Drug Report’ at URL unodc.org/wdr2016/). With higher-potency opioids, such as fentanyl and carfentanil, becoming ever-increasingly available within illicit drug markets (see Howard, “Why opioid overdose deaths seem to happen in spurts,” CNN Health, Feb. 8, 2017 at URL http://www.cnn.com/2017/02/08/health/opioids-overdose-deaths-epidemic-explainer), opioid-related deaths continue to be a problem across the United States.

Naloxone is a short-acting, non-addictive, opioid antagonist that is used for respiratory or central nervous system depression from opioid overdose (Sadove, M. S. et al., 1963, J. American Medical Assn. 183:666-668). Naloxone received US Food and Drug Administration (FDA) approval in 1971 and subsequently has become the standard first-line agent in the emergency setting for rapid reversal of accidental or intentional opioid overdose. However, naloxone undergoes extensive first-pass hepatic metabolism, making the oral route of delivery ineffective. Accordingly, it is most commonly injected intravenously for fastest action, though it can also be administered via intramuscular or subcutaneous injection, or nasal spray.

The delivery of naloxone intranasally or by injection can be complicated, particularly for individuals without medical training. For instance, the user must couple a needle (or an atomizer) to a syringe containing the naloxone formulation. After the device is prepared for delivery, the user then selects the region of the body in which the naloxone is to be delivered (e.g., into a vein, muscle or nasally), and deliver the naloxone. Accordingly, naloxone is often delivered by a healthcare provider in as controlled an environment as possible. However, because naloxone is often administered during an emergency situation, even experienced users may fail to deliver the drug effectively, whether via injection or intranasally.

Efforts have been made to develop improved ways to provide emergency personnel and laypeople with the ability to administer naloxone easily and effectively. For instance, the FDA recently approved two naloxone delivery vehicles: a pre-packaged nasal spray that does not require assembly and purportedly delivers a consistent dose; and a hand-held automatic injector that delivers an intramuscular injection of the drug. Both products are designed for use by laypersons, including family members and caregivers of opioid users at-risk for an opioid overdose or related emergency. However, both products are expensive, the auto-injector prohibitively so (recently reported at over $2,000 for a single dose; see Jacobs, “The EpiPen Isn't the Only Emergency Medicine Skyrocketing in Price,” Business Insider Aug. 29, 2016 at URL businessinsider.com/price-of-emergency-medecine-naloxone-narcan-skyrocketing). Moreover, both systems of administration still require accurately placing and physically introducing the drug into the recipient, e.g., by injection into a selected muscle or by projection into the nasal passage. In an emergency situation involving inexperienced or agitated users, this can result in incomplete or ineffective administration.

Thus, a need remains for improved methods and dosage forms for delivering opioid antagonists such as naloxone in an easy, reliable and cost-effective manner for use by emergency personnel and untrained users. Additionally, such dosage forms should be highly shelf stable under wide-ranging conditions for extended periods.

### SUMMARY OF THE INVENTION

Aspects of the present invention feature oral dissolvable films or oral disintegrating films for delivering an opioid antagonist to an individual. In addition, provided herein are methods for treating an individual using oral dissolvable films or oral disintegrating films containing an opioid antagonist.

One aspect of the invention features a dosage form for delivering an opioid antagonist to an individual in need of same that includes an oral dissolvable film containing the opioid antagonist in an amount effective to reduce or counteract the effect of an opioid overdose in the individual. In certain embodiments, the opioid antagonist is naloxone in free base form or in a pharmaceutically acceptable salt. In some aspects, dosage form includes between about 2 milligrams and about 25 milligrams of the naloxone. In other aspects, the dosage form dissolves completely within about 30 seconds and about 5 minutes following insertion into the oral cavity of the individual.

In some embodiments, the oral dissolvable film includes a polymer selected from hydroxypropylmethyl cellulose (HPMC), hydroxypropyl cellulose (HPC), pullulan, carboxymethyl cellulose (CMC), pectin, starch, polyvinyl acetate (PVA), sodium alginate, polyethylene oxide (PEO), polyvinyl pyrrolidone (PVP), and any combination thereof; particularly, the dosage form includes one or more of plasticizers, solubilizers, emulsifiers, sweetening and flavoring agents, coloring agents, saliva-stimulating agents, thickening agents, stabilizers, penetration enhancers, preservatives, antioxidants and buffering agents. In yet other embodiments, the dosage form includes naloxone or naloxone HCl, a base polymer, a plasticizer, a taste modifier, a solubilizer/emulsifier, a buffering agent and an optional penetration enhancer. In particular aspects, the base polymer is HPMC or PEO. In some embodiments, the plasticizer is propylene glycol and/or polyethylene glycol. In yet other embodiments, the taste modifier comprises a sweetener and/or a flavorant selected from Aspartame, peppermint oil or a combination thereof. In other aspects, the solubilizer/emulsifier is polyethoxylated castor oil. In still other aspects, the buffering agent is an organic acid. In yet other aspects, the optional penetration enhancer is selected from oleyl alcohol, polyoxyethylene 10 oleoyl ether, or a combination thereof.

Another aspect of the invention features a method of treating an individual experiencing an opioid overdose that includes the steps of determining that the individual is experiencing an opioid overdose; and administering into the oral cavity of the individual a dosage form comprising an oral dissolvable film that includes an opioid antagonist in an amount effective to reduce or counteract the effect of the opioid overdose in the individual.

In some embodiments, the opioid causing the overdose is one or a combination of heroin, fentanyl, carfentanil, oxycodone (oxycontin), oxymorphone, buprenorphine, methadone, sufentanil, alfentanil, morphine and hydrocodone. In other embodiments, the opioid antagonist comprises naloxone in free base form and/or as a pharmaceutically acceptable salt. In yet other embodiments, the dosage form comprises between about 2 mg and about 25 mg of naloxone. In other aspects, the method includes administering to the individual one or more subsequent doses of the dosage form.

Another aspect of the invention features a kit for treating an individual experiencing an opioid overdose. In such aspects, the kit includes a) a container in which is disposed one or more dosage units of a dosage form for delivering an opioid antagonist, comprising an oral dissolvable film that includes the opioid antagonist in an amount effective to reduce or counteract the effect of an opioid overdose in the patient; and b) instructions for administering one or more said dosage units to an individual experiencing an opioid overdose. In some embodiments, the container substantially protects the dosage units from exposure to one or more of light, heat and moisture.

Other features and advantages of the invention will be understood by the detailed description and examples that follow.

### BRIEF DESCRIPTION OF THE DRAWINGS

FIG. 1 is a graph showing the cumulative naloxone released from the 12 mg naloxone HCl ODF (12 mg NLX HCl, triangles) and the 12 mg naloxone free base ODF (12 mg NLX FB, squares). The y-axis depicts the cumulative naloxone released in mg, and the x-axis depicts time in minutes.

FIG. 2 is a graph showing the cumulative naloxone released from the 12 mg naloxone HCl ODF (12 mg NLX HCl, triangles) and the 12 mg naloxone free base ODF (12 mg NLX FB, squares). The y-axis depicts the percentage of naloxone released, and the x-axis depicts time in minutes.

FIG. 3 is a graph showing the cumulative naloxone permeation in an in vitro cultured human gingival tissue model for the 4 mg naloxone free base ODF (4 mg NLX FB, triangles), the 8 mg naloxone HCl ODF (8 mg NLX HCl, squares), and the 12 mg naloxone free base ODF (12 mg NLX FB, circles). The y-axis depicts the cumulative naloxone permeation in mg, and the x-axis depicts time in minutes.

FIG. 4 is a graph showing the cumulative naloxone permeation in an in vitro cultured human gingival tissue model for the 4 mg naloxone free base ODF (4 mg NLX FB, triangles), the 8 mg naloxone HCl ODF (8 mg NLX HCl, squares), and the 12 mg naloxone free base ODF (12 mg NLX FB, circles). The y-axis depicts the percentage of naloxone permeation, and the x-axis depicts time in minutes.

### DETAILED DESCRIPTION OF THE PREFERRED EMBODIMENTS

All percentages expressed herein are by weight of the total weight of the composition or mixture unless expressed otherwise. All ratios expressed herein are on a weight (w/w) basis unless expressed otherwise.

Ranges may be used herein in shorthand, to avoid having to list and describe each value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a method” or “a acid” includes a plurality of such “methods”, or “acids.” Likewise the terms “include”, “including”, and “or” should all be construed to be inclusive, unless such a construction is clearly prohibited from the context. Similarly, the term “examples,” particularly when followed by a listing of terms, is merely exemplary and illustrative and should not be deemed exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of.”

The methods and compositions and other advances disclosed herein are not limited to particular equipment or processes described herein because such equipment or processes may vary. Further, the terminology used herein is for describing particular embodiments only and is not intended to limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

The term “about” refers to the variation in the numerical value of a measurement, e.g., temperature, length, width, height, weight percentage, etc., due to typical error rates of the device used to obtain that measure. In one embodiment, the term “about” means within 5% of the reported numerical value.

Various aspects of the present invention feature a new and useful dosage form and method of administering opioid antagonists, such as naloxone, for the treatment of conditions arising from opioid overdose. Such overdose can be due to ingestion of one or more opioids, including but not limited to heroin, fentanyl, carfentanil, oxycodone (oxycontin), oxymorphone, buprenorphine, methadone, sufentanil, alfentanil, morphine and hydrocodone.

The novel dosage form comprises an oral dissolvable films (ODF) incorporating effective amounts of naloxone or other opioid antagonist. The dosage form provides rapid drug release properties and good mechanical properties (film integrity). The dosage form is placed in the patient's oral cavity, such as on or under the tongue, or along the inside of the cheek or between the tongue and cheek. It has been discovered that the dosage form thus administered is capable of delivering effective amounts of the opioid antagonist sufficiently rapidly to provide relief from the symptoms of opioid overdose. This was surprising in view of the fact that it was unclear whether sufficient opioid antagonist could be loaded into a suitably sized strip to deliver an effective dose of the antagonist.

Oral dissolvable films (ODF) are typically designed for oral administration, with the user placing the film on or under the tongue (sublingual) or along the inside of the cheek (buccal). ODF options allow the drug to bypass the first pass metabolism thereby making the drug more bioavailable. As the film dissolves, the drug can enter the blood stream enterically, buccally or sublingually. Evaluating the systemic transmucosal drug delivery, the buccal mucosa is the preferred region as compared to the sublingual mucosa.

Furthermore, active agents from such dosage forms can enter the body via the gastrointestinal system as well as transmucosally. Oral administration of naloxone has been shown to be ineffective due to the high first-pass effect, however, transmucosally it bypasses the first pass metabolism.

Any short-acting opioid antagonist can be used in the dosage forms described herein, including naloxone (see Barsan, W. G., 1989, Am. J. Emerg. Med. 7(2): 155-161) as salts or in their free base forms. In particular embodiments, naloxone hydrochloride is utilized, while in other embodiments, naloxone free base is utilized.

Oral dissolvable films (or oral disintegrating films) (ODFs) are well known in the art, and many are suitable for use in the present invention. As stated above, the ODFs of the present invention are designed for rapid drug release and good mechanical properties. Accordingly, various embodiments of the invention utilize single layer matrix films based on water-soluble polymers. Base polymers include, but are not limited to, hydroxypropylmethyl cellulose (HPMC), hydroxypropyl cellulose (HPC), pullulan, carboxymethyl cellulose (CMC), pectin, starch, polyvinyl acetate (PVA), sodium alginate, polyethylene oxide (PEO), polyvinyl pyrrolidone (PVP) and the like.

In certain embodiments, additional ingredients are incorporated into the films, including but not limited to plasticizers, solubilizers, emulsifiers, sweetening and flavoring agents, coloring agents, saliva-stimulating agents, thickening agents, stabilizers and modulators of delivery of the active pharmaceutical agent (API, i.e., the opioid antagonist) including penetration enhancers, preservatives, antioxidants and buffering agents.

For example, buffering agents or pH adjusting agents can include calcium carbonate, sodium bicarbonate, citric acid, sodium citrate anhydrous, tartaric acid, succinic acid, maleic acid, and fumaric acid. Antioxidants and preservatives include parabens, such as methyl paraben, ethyl paraben, propyl paraben, and butyl paraben, benzoic acid, sodium benzoate, sorbic acid, sodium sorbate, cetrimide, benzalkonium chlorise, cetylpyridium chloride, benzaethonium chloride, phenylmercuric nitrate, benzyl alcohol, phenylethyl alcohol, bronabol, chlorbutanol, chlorhexidine, butylated hydroxyanisole, butylated hydroxytoluene, tert-butyl hydroquinone, and 4-hydroxymethyl-2,6,-di-ter-butylphenol, to name a few.

In various embodiments, the ODF comprises a base polymer, a plasticizer, a taste modifier (sweetener and/or flavorant), a solubilizer/emulsifier, a buffering agent and an optional penetration enhancer. In embodiments particularly suitable to ODF-mediated delivery of naloxone, the base polymer comprises HPMC or a PEO. Exemplary HPMC base polymers include those having a methoxyl substitution percentage from about 20% wt to about 40% wt, a hydroxypropyl substitution percentage from about 3% wt to about 20% wt, and a viscosity from about 1 to about 20 mPa*s in water at 20° C.; particularly, those having methoxyl substitution percentage from about 25% wt to about 35% wt, a hydroxypropyl substitution percentage from about 5% wt to about 15% wt, and a viscosity from about 3 to about 7 mPa*s in water at 20° C. HPMC and PEO base polymers suitable for use in ODFs are commercially available (e.g., HPMC E5, METHOCEL™, Dow Pharma; POLYOX™ PEO, Dow Pharma). In particular embodiments, the plasticizer comprises one or more of a glycol, such as propylene glycol and polyethylene glycol (e.g., PEG-400). In certain embodiments, the taste modifiers include sweeteners (e.g., Aspartame) and flavors (e.g., peppermint oil). In certain embodiments, the solubilizer/emulsifier is a polyethoxylated castor oil (e.g., Kolliphor EL®, BASF Corporation). In certain embodiments, the buffering agent is an organic acid, e.g., citric acid. The optional penetration agent can be oleyl alcohol and/or polyoxyethylene 10 oleoyl ether (e.g., Brij 97/Oleth 10, Sigma).

In embodiments of the invention, the ODF can be of any size, shape and thickness suitable for loading a requisite amount of the opioid antagonist into a dosage unit. In certain embodiments, the dosage unit film is between about 1 to 3 cm by about 1 to 3 cm, e.g., 2.3×2.3 cm (or 1×1 inch). Film thickness is typically between about 5 and about 8 mils, though it may be thicker or thinner depending on the overall size and drug loading capacity. Likewise, film weight per dosage unit is typically between about 80 and about 120 mg.

The film type, size, shape, thickness and the like are selected to be loadable with about 2-25 mg of the opioid antagonist (e.g., naloxone HCl or naloxone free base) per dosage unit with a transmucosal tissue absorption of about 5% to about 50% of the opioid. In some embodiments, the minimum transmucosal absorption of the opioid is at least about 5%, 6%, 7%, 8%, 9%, 10%, 11%, 12%, 13%, 14%, 15%, 16%, 17%, 18%, 19%, 20%, 21%, 22%, 23%, 24%, 25%, or more. In certain embodiments, a dosage unit contains about 2 mg, or about 2.5 mg, or about 3 mg, or about 3.5 mg, or about 4 mg, or about 4.5 mg, or about 5 mg, or about 5.5 mg, or about 6 mg, or about 6.5 mg, or about 7 mg, or about 7.5 mg, or about 8 mg, or about 8.5 mg, or about 9 mg, or about 9.5 mg, or about 10 mg, or about 10.5 mg, or about 11 mg, or about 11.5 mg, or about 12 mg, or about 12.5 mg, or about 13 mg, or about 13.5 mg, or about 14 mg, or about 14.5 mg, or about 15 mg, or about 15.5 mg, or about 16 mg, or about 16.5 mg, or about 17 mg, or about 17.5 mg, or about 18 mg, or about 18.5 mg, or about 19 mg, or about 19.5 mg, or about 20 mg, or about 20.5 mg, or about 21 mg, or about 21.5 mg, or about 22 mg, or about 22.5 mg, or about 23 mg, or about 23.5 mg, or about 24 mg, or about 24.5 mg, or about 25 mg of naloxone free base or naloxone HCl. For instance, in a particular embodiment, the dosage unit contains up to about 20 mg of naloxone free base or naloxone HCl, assuming a minimum 5% transmucosal absorption. The ODF is designed such that it dissolves and complete drug release is effected within less than a minute to about 5 minutes. More particularly, the ODF dissolves within about 0.5 minute, or about 1 minute, or about 1.5 minutes, or about 2 minutes, or about 2.5 minutes, or about 3 minutes, or about 3.5 minutes, or about 4 minutes, or about 4.5 minutes, or about 5 minutes.

ODFs comprising an API such as an opioid antagonist can be manufactured by one or a combination of processes known in the art. These include solvent casting, semisolid casting, hot-melt extrusion (HME), solid-dispersion extrusion, and rolling. The most commonly used methods of film manufacturing are solvent casting and HME (see, e.g., Mishra, R. & A. Amin, 2011, Pharmaceutical Technology 35 (1) (pharmtech.com)).

The opioid antagonist ODF is preferably formulated using the solvent-casting method, followed by oven drying. In certain embodiments, the ingredients are dissolved in a suitable solvent, preferably a Class 3 residual solvent, e.g., water, alcohol, acetone and combinations thereof. Certain of the components can be dissolved separately and then combined in the main solution. The solution is deaerated, e.g., under vacuum, then cast as a film and heated to dryness.

Once the films are dried, they can be cut into suitable sizes and shapes, and then packaged. The packaging container should provide sufficient mechanical strength to protect the film during shipping and from external factors such as temperature and humidity. Depending upon the characteristics of the film, single-unit containers or multiple-unit dispensers can be selected.

Once fabricated, the ODF can be evaluated by various parameters such as thickness, the mechanical properties of the film (e.g., tensile strength, percentage elongation and elastic modulus), folding endurance, assay of drug content, in-vitro disintegration/dissolution, surface morphology and taste, to name a few. The skilled artisan is familiar with techniques for measuring such parameters (see, e.g., Mishra & Amin, 2011, supra).

Clinical parameters of the ODF are also measured in accordance with known techniques. For instance, comparative bioequivalence between an injectable formulation and an ODF comprising the opioid antagonist can be evaluated. Safety, tolerability, and efficacy features can also be demonstrated in pre-clinical (e.g., hamster cheek model) and/or clinical studies.

Another aspect of the invention features methods of using the opioid antagonist in an ODF dosage form. In certain embodiments, the ODF is placed in the oral cavity of the subject, such as on or under the tongue, or along the inside of the cheek or between the tongue and cheek, and allowed to dissolve completely. The ODF can be administered to anyone in need of an opioid antidote, such as is well known in the art for injectable versions of such agents.

In some embodiments, more than one ODF dosage unit may be administered sequentially, for instance when the individual administering the ODF determines that additional dosing is needed to more completely counteract the effect of the overdose.

In particular embodiments, the opioid antagonist-containing ODF dosage form dissolves within about 2-5 minutes. The dosage unit should be allowed to dissolve completely before another dosage unit is administered.

The following nonlimiting examples are provided to describe the invention in greater detail.

### Example 1

A batch of ODFs containing 4 mg Naloxone HCl per dosage unit was prepared in accordance with the following formulation and procedure.

TABLE 1

| Ingredient | Function | wet g | dry g | dry % |
|---|---|---|---|---|
| Propylene | plasticizer | 2.00 | 2 | 12.3% |
| Glycol | | | | |
| PEG-400 | plasticizer | 2.00 | 2 | 12.3% |
| Peppermint Oil | flavoring agent | 0.25 | 0.25 | 1.5% |
| Kolliphor EL ® | solubilizer | 0.30 | 0.3 | 1.8% |
| Ethanol | solvent | 10.00 | | |
| Water | solvent | 25.00 | | |
| HPMC E5 | matrix polymer | 10.50 | 10.5 | 64.7% |
| Aspartame | sweetener | 0.25 | 0.25 | 1.5% |
| Citric Acid | buffering agent | 0.25 | 0.25 | 1.5% |
| Naloxone HCl | active agent | 0.67 | 0.67 | 4.1% |
| total | | 51.22 | 16.22 | 100% |

solids % 31.7%
solvents % 68.3%

Liquid components (propylene glycol, PEG-400, Kolliphor EL, peppermint oil, water and ethanol) were weighed into a 100 mL beaker and mixed for 5 minutes. The HPMC E5 was weighed and mixed into the liquid components. Mixing was continued until the solid was dissolved (about 15-20 min). Aspartame, citric acid and naloxone HCl were weighed and added to the solution. Mixing was continued until those solid components were dissolved (about 10 minutes). The solution was allowed to stand for about 2-3 hours to allow foam to settle.

The solution was cast at 30 mils onto 3M Scotchpak 1022 release liners (non-release side). The casts were allowed to air dry for 20-30 minutes, then oven dried 10 min at 75° C. The film was peeled off the liner and die-cut into 1″ by 1″ squares. Individual film squares were packaged into pouches.

### Example 2

Two naloxone HCl ODFs (5 mg naloxone per dosage unit) were prepared in accordance with the method described in Example 1. The formulations are shown in Table 2. The films were observed and compared for color, texture, wet-out on release liner (non-release side; an indicator measuring ease of spreading and removal), and time to complete dissolution. For the dissolution measurement, one dosage unit (1″×1″ film) was placed in 90 mL water at room temperature and stirred at 200 rpm.

TABLE 2

| Class | Ingredient* | A | B |
|---|---|---|---|
| API | Naloxone HCl | 6.5% | 6.5% |
| Matrix Polymer | HPMC E5 | | 67.7% |
| | PolyOx (PEO) | 67.7% | |
| Plasticizer | PEG-400 | 9.7% | 9.7% |
| | Propylene Glycol | 9.7% | 9.7% |
| Taste Modifier | Aspartame | 1.6% | 1.6% |
| | Peppermint Oil | 1.6% | 1.6% |
| Buffering agent | Citric Acid | 1.3% | 1.3% |
| Emulsifying agent | Kolliphor EL | 1.9% | 1.9% |
| Notes | Total | 100.0% | 100.0% |
| | Wet-out on liner | good | good |
| | Film color | hazy whitish | clear |
| | Film texture | Very good, flexible | good** |
| | Dissolution (min) | 1 | 2 |

*Solvents: ethanol/water = 2/5; solids = 25 to 29%; wet casting at 30 mils.
**can be improved with more plasticizers

### Example 3

Three naloxone HCl ODFs (4 mg naloxone per dosage unit) were prepared in accordance with the method described in Example 1. The formulations are shown in Table 3.

TABLE 3

| | | Formulation ID | | |
|---|---|---|---|---|
| Ingredient | Function | 170119 | 170120A | 170120B |
| Propylene Glycol | plasticizer | 12.3% | 11.6% | 11.6% |
| PEG-400 | plasticizer | 12.3% | 11.7% | 11.7% |
| Peppermint Oil | flavoring agent | 1.5% | 1.5% | 1.5% |
| Kolliphor EL | solubilizer | 1.8% | 1.80% | 1.80% |
| Oleyl alcohol | penetration enhancer | | 5.10% | |
| Brij 97/Oleth 10 | penetration enhancer | | | 5.10% |
| HPMC E5 | matrix polymer | 64.7% | 61.3% | 61.3% |
| Aspartame | sweetener | 1.5% | 1.5% | 1.5% |
| Citric Acid | buffering agent | 1.5% | 1.5% | 1.5% |
| Naloxone HCl | active ingredient | 4.1% | 4.0% | 4.0% |
| | | 100.0% | 100% | 100% |

Each dosage unit (2.3 cm×2.3 cm or 1″×1″) weighs about 100 mg, and contains 4 mg of naloxone HCl. When one film was placed in 90 mL of water, with stirring speed at 200 rpm, at room temperature, time to complete dissolution was less than two minutes. The film was observed to be flexible, yet tough (not brittle).

### Example 4

A batch of ODFs containing 8 mg Naloxone HCl per dosage unit was prepared in accordance with the following formulation and procedure. The formulation is shown in Table 4.

TABLE 4

| Ingredient | Function | wet g | dry g | dry % |
|---|---|---|---|---|
| Propylene Glycol | plasticizer | 2.00 | 2.00 | 11.8% |
| PEG-400 | plasticizer | 2.00 | 2.00 | 11.8% |
| Peppermint Oil | flavoring agent | 0.25 | 0.25 | 1.5% |
| Kolliphor EL ® | solubilizer | 0.30 | 0.30 | 1.8% |
| Ethanol | solvent | 10.00 | | |
| Water | solvent | 25.00 | | |
| HPMC E5 | matrix polymer | 10.50 | 10.50 | 62.2% |
| Aspartame | sweetener | 0.25 | 0.25 | 1.5% |
| Citric Acid | buffering agent | 0.25 | 0.25 | 1.5% |
| Naloxone HCl | active agent | 1.34 | 1.34 | 7.9% |
| total | | 51.89 | 16.89 | 100% |

solids % 32.5%
Naloxone mg in 100 mg

Liquid components (propylene glycol, PEG-400, Kolliphor EL, peppermint oil, water and ethanol) were weighed into a 100 mL beaker and mixed for 5 minutes. The HPMC E5 was weighed and added to the liquid components. Mixing was continued until the solid was dissolved (about 15-20 min). Aspartame, citric acid and naloxone HCl were weighed and mixed into the solution. Mixing was continued until those solid components were dissolved (about 10 minutes). The solution was allowed to stand for about 2-3 hours to allow foam to settle.

The solution was cast at 30 mils onto 3M Scotchpak 1022 release liners (non-release side). The casts were allowed to air dry for 20-30 minutes, then oven dried 10 min at 75° C. In some examples, the casts were allowed to air dry overnight. The film was peeled off the liner and die-cut into 1″ by 1″ squares. Individual film squares were packaged into pouches.

### Example 5

A batch of ODFs containing 4 mg Naloxone Free Base per dosage unit was prepared in accordance with the method described in Example 4. The formulation is shown in Table 5.

TABLE 5

| Ingredient | Function | wet g | dry g | dry % |
|---|---|---|---|---|
| Propylene Glycol | plasticizer | 2.00 | 2.00 | 12.3% |
| PEG-400 | plasticizer | 2.00 | 2.00 | 12.3% |
| Peppermint Oil | flavoring agent | 0.25 | 0.25 | 1.5% |
| Kolliphor EL ® | solubilizer | 0.30 | 0.30 | 1.8% |
| Ethanol | solvent | 10.00 | | |
| Water | solvent | 25.00 | | |
| HPMC E5 | matrix polymer | 10.50 | 10.50 | 64.7% |
| Aspartame | sweetener | 0.25 | 0.25 | 1.5% |
| Citric Acid | buffering agent | 0.25 | 0.25 | 1.5% |
| Naloxone Base | active agent | 0.67 | 0.67 | 4.1% |
| total | | 51.22 | 16.22 | 100% |

solids % 31.7%
Naloxone mg in 95 mg

### Example 6

A batch of ODFs containing 4 mg Naloxone Free Base per dosage unit was prepared in accordance with the following formulation and procedure. The formulation is shown in Table 6.

TABLE 6

| Ingredient | Function | wet g | dry g | dry % |
|---|---|---|---|---|
| Propylene Glycol | plasticizer | 1.99 | 1.99 | 11.6% |
| PEG-400 | plasticizer | 2.00 | 2.00 | 11.7% |
| Peppermint Oil | flavoring agent | 0.25 | 0.25 | 1.5% |
| Oleyl alcohol | penetration enhancer | 0.87 | 0.87 | 5.1% |
| Kolliphor EL ® | solubilizer | 0.31 | 0.31 | 1.8% |
| Ethanol | solvent | 10.21 | | |
| Water | solvent | 25.19 | | |
| HPMC E5 | matrix polymer | 10.50 | 10.50 | 61.3% |
| Aspartame | sweetener | 0.26 | 0.26 | 1.5% |
| Citric Acid | buffering agent | 0.26 | 0.26 | 1.5% |
| Naloxone Base | active agent | 0.68 | 0.68 | 4.0% |
| total | | 52.52 | 17.12 | 100% |

solids % 32.6%

Propylene glycol, PEG-400, Kolliphor EL, peppermint oil, oleyl alcohol, water and ethanol) were weighed into a 100 mL beaker and mixed for 5 minutes. The HPMC E5 was weighed and added to the liquid components. Mixing was continued until the solid was dissolved (about 15-20 min). Aspartame, citric acid and naloxone HCl were weighed and mixed into the solution. Mixing was continued until those solid components were dissolved (about 10 minutes). The solution was allowed to stand for about 2 hours to allow foam to settle.

The solution was cast at 30 mils onto 3M Scotchpak 1022 release liners (non-release side). The casts were allowed to air dry overnight. The film was peeled off the liner and die-cut into 1″ by 1″ squares. Individual film squares were packaged into pouches.

### Example 7

A batch of ODFs containing 12 mg Naloxone Free Base per dosage unit was prepared in accordance with the following formulation and procedure. The formulation is shown in Table 7.

TABLE 7

| Ingredient | Function | wet g | dry g | dry % |
|---|---|---|---|---|
| Propylene Glycol | plasticizer | 2.00 | 2.00 | 10.9% |
| PEG-400 | plasticizer | 2.00 | 2.00 | 10.9% |
| Peppermint Oil | flavoring agent | 0.25 | 0.25 | 1.4% |
| Brij 97/Oleth 10 | penetration enhancer | 0.85 | 0.85 | 4.6% |
| Ethanol | solvent | 10.00 | | |
| Water | solvent | 25.00 | | |
| HPMC E5 | matrix polymer | 10.50 | 10.50 | 57.4% |
| Aspartame | sweetener | 0.25 | 0.25 | 1.4% |
| Citric Acid | buffering agent | 0.25 | 0.25 | 1.4% |
| Naloxone Base | active agent | 2.20 | 2.20 | 12.0% |
| total | | 53.30 | 18.30 | 100% |

solids % 34.3%

Propylene glycol, PEG-400, peppermint oil, Brij 97/Oleth 10, water and ethanol) were weighed into a 100 mL beaker and mixed for 5 minutes. The HPMC E5 was weighed and added to the liquid components. Mixing was continued until the solid was dissolved (about 15-20 min). Aspartame, citric acid and naloxone HCl were weighed and mixed into the solution. Mixing was continued until those solid components were dissolved (about 10 minutes). The solution was allowed to stand for about 1-2 hours to allow foam to settle.

The solution was cast at 30 mils onto the 3M Scotchpak 1022 release liners (non-release side). The casts were allowed to air dry overnight. The film was peeled off the liner and die-cut into 1″ by 1″ squares. Individual film squares were packaged into pouches.

### Example 8

The drug release profile was assessed for two ODF formulations (i.e., 12 mg naloxone free base and 12 mg naloxone HCL). The formulations were prepared as in Example 7 and are summarized in Tables 8 and 9.

TABLE 8
12 mg Naloxone HCl Formulation.

| | wet g | dry g | dry % | Actual |
|---|---|---|---|---|
| Propylene Glycol | 2.00 | 2.00 | 10.9% | 2.00 |
| PEG-400 | 2.00 | 2.00 | 10.9% | 2.03 |
| Peppermint Oil | 0.25 | 0.25 | 1.4% | 0.25 |
| Brij 97/Oleth 10 | 0.85 | 0.85 | 4.6% | 0.88 |
| Ethanol | 10.00 | | | 10.51 |
| Water | 25.00 | | | 25.00 |
| HPMC E5 | 10.50 | 10.5 | 57.4% | 10.55 |
| Aspartame | 0.25 | 0.25 | 1.4% | 0.26 |
| Citric Acid | 0.25 | 0.25 | 1.4% | 0.26 |
| Naloxone HCl | 2.20 | 2.2 | 12.0% | 2.23 |
| total | 53.30 | 18.30 | 100% | |

solids % 34.3%

TABLE 9
12 mg Naloxone Free Base Formulation.

| | wet g | dry g | dry % | Actual |
|---|---|---|---|---|
| Propylene Glycol | 2.00 | 2 | 10.9% | 2.01 |
| PEG-400 | 2.00 | 2 | 10.9% | 2.02 |
| Peppermint Oil | 0.25 | 0.25 | 1.4% | 0.26 |
| Brij 97/Oleth 10 | 0.85 | 0.85 | 4.6% | 0.87 |
| Ethanol | 10.00 | | | 10.05 |
| Water | 25.00 | | | 25.08 |
| HPMC E5 | 10.50 | 10.5 | 57.4% | 10.52 |
| Aspartame | 0.25 | 0.25 | 1.4% | 0.25 |
| Citric Acid | 0.25 | 0.25 | 1.4% | 0.25 |
| Naloxone base | 2.20 | 2.2 | 12.0% | 2.21 |
| total | 53.30 | 18.30 | 100% | |

solids % 34.3%

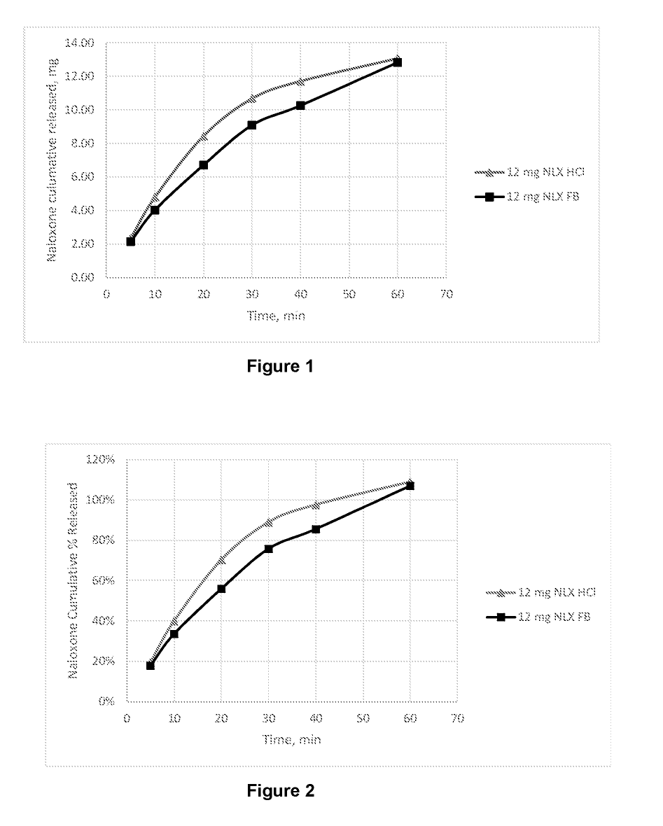

To measure drug dissolution, 12 mL, 1.767 cm² Franz cells at 37° C. were used (Logan Instruments Corp., Somerset, N.J., USA). Each ODF sample was die-cut into 0.5 inch (1.27 cm) diameter discs (area, 1.267 cm²) and placed onto 25 mm diameter Whatman membrane discs (GE Healthcare) of the Franz cells. The membrane discs had a pore size of 0.45 μm and were comprised of NC45 cellulose nitrate. Phosphate buffer solution pH 6.8 was added into each cell. The cells were sampled at 5 min, 10 min, 20 min, 30 min, 40 min, and 60 min. At each time point, 1.5 mL of the receptor medium was drawn up and assayed by HPLC in a 5 μm, 4.6×50 mm KINETEX C18 column (Phenomenex, Inc., Torrance, Calif., USA) using art standard protocols. The results of this assay were normalized based on a 1 inch (2.54 cm)×1 inch (2.54 cm) film. Briefly, the mobile phase comprised 50 mM NH₄Ac/Acetonitrile at 88/22. The flow rate was 1.0 mL/min and the column temperature was about 40° C. with a UV of 220 nm and a retention time of 1.8 min. Both ODF formulations exhibited complete drug dissolution by 60 minutes. The results are shown in Tables 10 and 11 and FIGS. 1 and 2.

TABLE 10
Cumulative Release of Naloxone (mg)

| | 12 mg | 12 mg Naloxone |
|---|---|---|
| Time, min | Naloxone HCl | free base FB |
| 5 | 2.34 | 2.14 |
| 10 | 4.80 | 4.03 |
| 20 | 8.44 | 6.71 |
| 30 | 10.68 | 9.09 |
| 40 | 11.72 | 10.27 |
| 60 | 13.08 | 12.84 |

TABLE 11
Cumulative Release of Naloxone (%)

| | | 12 mg |
|---|---|---|
| | 12 mg Naloxone | Naloxone free |
| Time, min | HCl | base |
| 5 | 19.5% | 17.9% |
| 10 | 40.0% | 33.6% |
| 20 | 70.4% | 55.9% |
| 30 | 89.0% | 75.8% |
| 40 | 97.6% | 85.6% |
| 60 | 109.0% | 107.0% |

### Example 9

Three ODF formulations (i.e., 4 mg naloxone free base, 8 mg naloxone HCL, and 12 mg naloxone free base) were assessed for drug permeation efficacy at 15 min, 30 min, 60 min, and 180 min using cultured human gingival tissues. The three ODF formulation compositions are summarized in Table 12.

TABLE 12
ODF Formulation Compositions

| | | Formulation ID | | |
|---|---|---|---|---|
| | | 4 mg | 8 mg | 12 mg |
| | | Naloxone | Naloxone | Naloxone |
| | | free base | HCl | free base |
| Ingredient Name | Function | Lot# 170202B | Lot# 170119 | Lot# 170203 |
| Propylene Glycol | plasticizer | 11.8% | 11.8% | 10.9% |
| PEG-400 | plasticizer | 11.8% | 11.8% | 10.9% |
| Peppermint Oil | flavoring agent | 1.5% | 1.5% | 1.4% |
| Kolliphor EL | solubilizer | | 1.8% | |
| Oleyl alcohol | penetration enhancer | 5.20% | | |
| Brij 97/Oleth 10 | penetration enhancer | | | 4.60% |
| HPMC E5 | matrix polymer | 62.5% | 62.2% | 57.4% |
| Aspartame | sweetener | 1.5% | 1.5% | 1.4% |
| Citric Acid | buffering agent | 1.5% | 1.5% | 1.4% |
| Naloxone HCl | active ingredient | | 8.0% | |
| Naloxone FB | active ingredient | 4.0% | | 12.0% |
| | | 100% | 100% | 100% |

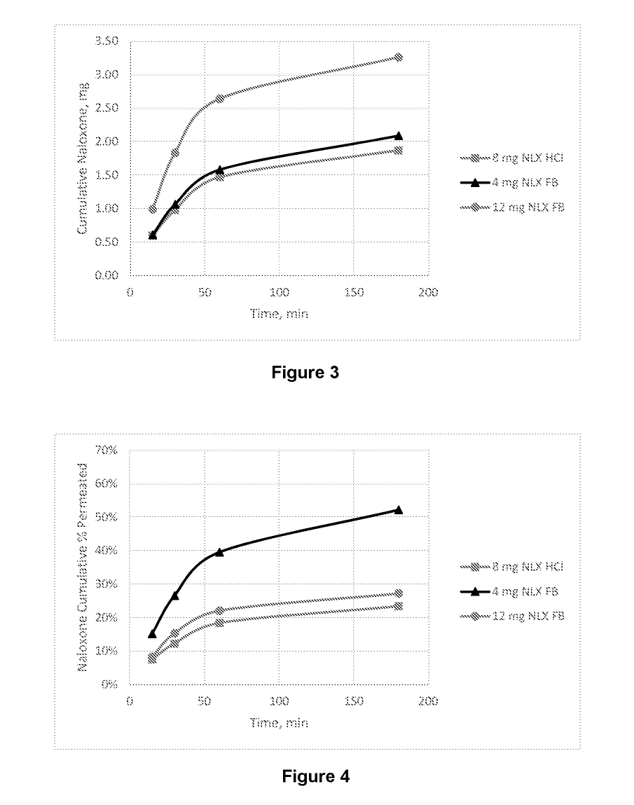

Each ODF sample was die-cut into 1 inch (2.54 cm) diameter discs (area, 5.067 cm²). To simulate the oral mucosa, an in vitro oral mucosa 3D model produced from primary human gingival keratinocytes was used (SkinAxis, LLC, North Brunswick, N.J., USA). Briefly, the cultured human gingival tissues were incubated in 6-well plates at 37° C. in a CO₂ incubator. To prepare the receiver fluid, 0.3 mL of prewarmed phosphate buffered solution at pH 6.8 was pipetted into each well. Next, 0.5 mL of donor solution or the die-cut discs were placed onto the tissues. The plates were returned to the incubator for 15 minutes. The tissues were moved to the next wells and the receiver media was collected. This was repeated for the remaining time points. The receiver media was analyzed by HPLC in a 5 μm, 4.6×50 mm KINETEX C18 column (Phenomenex, Inc., Torrance, Calif., USA) using art standard protocols. Briefly, the mobile phase comprised 50 mM NH₄Ac/Acetonitrile at 88/22. The flow rate was 1.0 mL/min and the column temperature was about 40° C. with a UV of 220 nm and a retention time of 1.8 min. The permeation enhancer oleyl alcohol increased the permeation percentage of the naloxone. The cumulative amount of Naloxone permeated is summarized in Tables 13 and 14 and FIGS. 3 and 4.

TABLE 13
Cumulative Naloxone Permeation (mg)

| | | | 12 mg |
|---|---|---|---|
| | 8 mg Naloxone | 4 mg Naloxone | Naloxone free |
| Time, min | HCl | free base | base |
| 15 | 0.60 | 0.61 | 0.99 |
| 30 | 0.98 | 1.06 | 1.83 |
| 60 | 1.47 | 1.58 | 2.64 |
| 180 | 1.87 | 2.09 | 3.26 |

TABLE 14
Cumulative Naloxone Permeation (%)

| | | | 12 mg |
|---|---|---|---|
| | 8 mg Naloxone | 4 mg Naloxone | Naloxone free |
| Time, min | HCl | free base | base |
| 15 | 7.5% | 15.1% | 8.3% |
| 30 | 12.2% | 26.6% | 15.3% |
| 60 | 18.4% | 39.5% | 22.0% |
| 180 | 23.4% | 52.2% | 27.2% |

The present invention is not limited to the embodiments described and exemplified above. It is capable of variation and modification within the scope of the appended claims.

1. A dosage form for delivering an opioid antagonist to an individual in need of same, comprising an oral dissolvable film that includes the opioid antagonist in an amount effective to reduce or counteract the effect of an opioid overdose in the individual.

2. The dosage form of claim 1, wherein the opioid antagonist is naloxone in free base form or in a pharmaceutically acceptable salt.

3. The dosage form of claim 2, comprising between about 2 milligrams and about 25 milligrams of the naloxone.

4. The dosage form of claim 1, which dissolves completely within about 30 seconds and about 5 minutes following insertion into the oral cavity of the individual.

5. The dosage form of claim 1, wherein the oral dissolvable film comprises a polymer selected from hydroxypropylmethyl cellulose (HPMC), hydroxypropyl cellulose (HPC), pullulan, carboxymethyl cellulose (CMC), pectin, starch, polyvinyl acetate (PVA), sodium alginate, polyethylene oxide (PEO), polyvinyl pyrrolidone (PVP), and any combination thereof.

6. The dosage form of claim 5, further comprising one or more of plasticizers, solubilizers, emulsifiers, sweetening and flavoring agents, coloring agents, saliva-stimulating agents, thickening agents, stabilizers, penetration enhancers, preservatives, antioxidants and buffering agents.

7. The dosage form of claim 1, comprising naloxone or naloxone HCl, a base polymer, a plasticizer, a taste modifier, a solubilizer/emulsifier, a buffering agent and an optional penetration enhancer.

8. The dosage form of claim 7, wherein the base polymer is HPMC or PEO.

9. The dosage form of claim 7, wherein the plasticizer is propylene glycol and/or polyethylene glycol.

10. The dosage form of claim 7, wherein the taste modifier comprises a sweetener and/or a flavorant selected from Aspartame, peppermint oil or a combination thereof.

11. The dosage form of claim 7, wherein the solubilizer/emulsifier is polyethoxylated castor oil.

12. The dosage form of claim 7, wherein the buffering agent is an organic acid.

13. The dosage form of claim 7, wherein the optional penetration enhancer is selected from oleyl alcohol, polyoxyethylene 10 oleoyl ether, or a combination thereof.

14. A method of treating an individual experiencing an opioid overdose, comprising:
a) determining that the individual is experiencing an opioid overdose; and
b) administering into the oral cavity of the individual a dosage form comprising an oral dissolvable film that includes an opioid antagonist in an amount effective to reduce or counteract the effect of the opioid overdose in the individual.

15. The method of claim 14, wherein the opioid causing the overdose is one or a combination of heroin, fentanyl, carfentanil, oxycodone (oxycontin), oxymorphone, buprenorphine, methadone, sufentanil, alfentanil, morphine and hydrocodone.

16. The method of claim 14, wherein the opioid antagonist comprises naloxone in free base form and/or as a pharmaceutically acceptable salt.

17. The method of claim 14, wherein the dosage form comprises between about 2 mg and about 25 mg of naloxone.

18. The method of claim 14, comprising administering to the individual one or more subsequent doses of the dosage form.

19. A kit for treating an individual experiencing an opioid overdose, comprising
a) a container in which is disposed one or more dosage units of a dosage form for delivering an opioid antagonist, comprising an oral dissolvable film that includes the opioid antagonist in an amount effective to reduce or counteract the effect of an opioid overdose in the patient; and
b) instructions for administering one or more said dosage units to an individual experiencing an opioid overdose.

20. The kit of claim 19, wherein the container substantially protects the dosage units from exposure to one or more of light, heat and moisture.
